(19) United States
(12) Patent Application Publication
(10) Pub. No.: US 2022/0161441 A1
(43) Pub. Date: May 26, 2022

(54) ROBOT BODY CARE SYSTEM, ROBOT BODY CARE METHOD, AND ROBOT BODY CARE PROGRAM

(71) Applicant: Riccoh Co., Ltd., Tokyo (JP)

(72) Inventor: Yoshiyuki HARASHIMA, Tokyo (JP)

(73) Assignee: Riccoh Co., Ltd., Tokyo (JP)

(21) Appl. No.: 17/648,025

(22) Filed: Jan. 14, 2022

Related U.S. Application Data
(63) Continuation of application No. PCT/JP2020/027492, filed on Jul. 15, 2020.

(30) Foreign Application Priority Data
Jul. 17, 2019 (JP) 2019-132267

(51) Int. Cl. B25J 11/00; B25J 13/08; B25J 19/02; A61H 15/00; A61H 23/02

(52) U.S. Cl. CPC B25J 11/008; B25J 13/081; B25J 19/028; A61H 2201/1659; A61H 23/02; A61H 2201/1207; A61H 15/0078

(57) ABSTRACT

A robot body care system according to the present disclosure includes: a body care device which performs body care by applying physical stimulation to a user; and an information processing server including a reception unit that receives, from the outside as treatment information, a treatment method indicating a way of applying stimulation to the portions of the body of the user when body care is performed on the user, a command value generation unit that generates treatment command values as electrical signals on the basis of the treatment information entered into the reception unit, and a transmission unit that transmits the treatment command values to the body care device.

### CROSS-REFERENCE TO RELATED APPLICATIONS

This application is a continuation International Patent Application No. PCT/JP2020/027492 filed Jul. 15, 2020, which claims the benefit of priority to Japanese Patent Application No. 2019-132267 filed Jul. 17, 2019, the content of each of which is incorporated herein by reference in its entirety.

### BACKGROUND

### 1. Technical Field

The present disclosure relates to a robot body care system, a robot body care method, and a robot body care program.

### 2. Description of Related Art

The businesses related to body care in which physical stimulation is applied to a user through various treatment methods have been conducted conventionally, and in the businesses, training of practitioners has been actively conducted. For example, patent document 1 below discloses a system that allows a practitioner to record his/her body care practice performance to train the practitioner.

[Patent Document 1] Japanese Laid-Open Patent Publication No. 2009-42924

### SUMMARY

However, in the training of practitioners utilizing the technology described in patent document 1 above, time is needed for the practitioners to become proficient in the technology, and securing personnel has been a problem. That is, in such a labor-intensive service, it was difficult to continue to provide stable quality of body care over a long period of time due to the large percentage of dependence on the practitioners.

Therefore, it is an object of the present disclosure to provide a robot body care system capable of effectively utilizing the know-how possessed by various practitioners, simplifying labor-intensive services such as body care services, and stabilizing the quality of the body care.

A robot body care system according to the present disclosure includes: a body care device that performs body care by applying physical stimulation to a user; and an information processing server including a reception unit that receives from the outside a treatment method indicating a way of applying stimulation to the portions of the body of the user when body care is performed on the user as treatment information, a command value generation unit that generates treatment command values as electrical signals on the basis of the treatment information entered into the reception unit, and a transmission unit that transmits the treatment command values to the body care device.

The body care system also includes a history recording unit that records history information indicating a history in which the treatment command values generated by the command value generation unit are used; an evaluation input unit in which evaluation information indicating an evaluation of the treatment command values used is entered by the user; and a command value evaluation unit that evaluates the popularity of the treatment command values on the basis of the history information recorded in the history recording unit and the evaluation information entered into the evaluation input unit.

Further, the evaluation information may be entered into the evaluation input unit by a plurality of users.

Moreover, the system may further include a command value proposal unit that proposes highly popular treatment command values to any one of a plurality of users on the basis of the evaluation results by the command value evaluation unit.

Furthermore, the reception unit may include a business operator identification unit that receives the treatment information from the body care related business operators, and identifies, on the basis of the evaluation results by the command value evaluation unit, the body care related business operator that has entered the highly popular treatment information as an excellent business operator.

In addition, a reward may be paid to an excellent business operator identified by the business operator identification unit.

In addition, in order to solve the above problem, the robot body care method of the present disclosure causes a computer to perform the steps of: receiving from an outside a treatment method that indicates a way of applying stimulation to the portions of the body of the user when the body care is performed on the user as treatment information; generating treatment command values as electrical signals on the basis of the treatment information entered into a reception unit; transmitting the treatment command values to a body care device that applies physical stimulation to the user to perform the body care; and performing the body care by the body care device using the treatment command values.

In addition, in order to solve the above problem, the robot body care program of the present disclosure causes a computer to execute the functions of: receiving from an outside a treatment method that indicates a way of applying stimulation to the portions of the body of the user when the body care is performed on the user as treatment information; generating treatment command values as electrical signals on the basis of the treatment information entered into a reception unit; transmitting the treatment command values to a body care device that applies physical stimulation to the user to perform the body care; and performing the body care by the body care device using the treatment command values.

In the robot body care system according to the present disclosure, a command value generation unit generates treatment command values from treatment information received by the reception unit from outside. The body care device then performs body care on the user on the basis of the treatment command values. This eliminates the need for the practitioner to directly perform the body care, and allows the practitioner's know-how regarding the body care to be quantitatively managed as treatment command values. This effectively utilizes the know-how possessed by various practitioners, simplifies the labor-intensive services such as body care services, and stabilizes the quality of the body care.

### BRIEF DESCRIPTION OF THE DRAWINGS

FIG. 1 is a system diagram illustrating a configuration example of a robot body care system according to the present disclosure.

FIG. 2 is a block diagram illustrating a configuration of an information processing terminal in the robot body care system shown in FIG. 1.

FIG. 3 is a block diagram illustrating a configuration of an information processing server in the robot body care system shown in FIG. 1.

FIG. 4 is a schematic diagram illustrating an overview of the process by which a command value generation unit shown in FIG. 1 generates treatment command values.

FIG. 5 is a flowchart illustrating a behavior of body care by a body care device shown in FIG. 1.

FIG. 6 is a flowchart showing a processing flow in the information processing server.

### DETAILED DESCRIPTION OF EMBODIMENTS

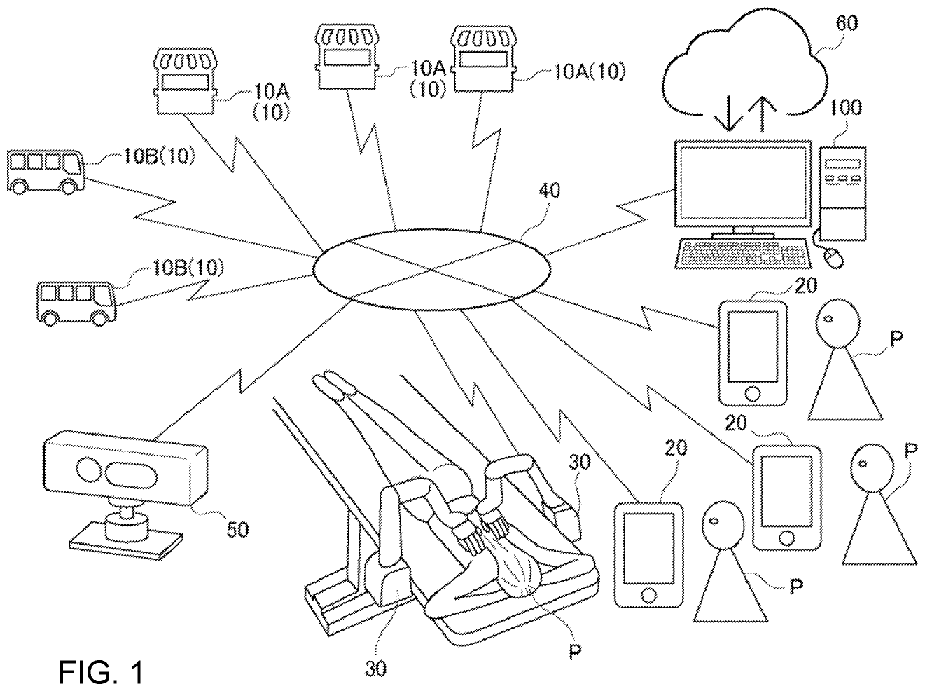

A robot body care system 1 according to one embodiment of the present disclosure will be described in detail below with reference to the drawings. FIG. 1 is a system diagram illustrating a configuration example of the robot body care system 1. As shown in FIG. 1, the robot body care system 1 includes an information processing server 100 that serves as a main server. The information processing server 100 is connected to external terminals via the network 40. The external terminals include a plurality of information processing terminals 20 possessed by corresponding users P, a body care device 30, a camera 50, and cloud 60. The network 40 is also connected to a plurality of body care related business operators 10.

The body care related business operators 10 (hereinafter, simply referred to as business operators 10) include, for example, business operators that provide various types of body care, such as esthetic salons, body care stores, osteopathic clinics, and Chiropractic clinics. Here, as shown in FIG. 1, the business operator 10 may be a storefront business operator 10A that conducts business at a fixed store, or a mobile business operator 10B that conducts business in a service vehicle loaded with equipment. In the case of the mobile business operator 10B, the body care device 30 is loaded inside the service vehicle, and the user receiving the body care can, for example, use a smartphone application to call the service vehicle to the desired point and receive the body care service in the service vehicle. The mobile operator 10B may be a manned vehicle with an operator driving the service vehicle and guiding the body care service, or it may be an unmanned vehicle that is driven automatically.

The business operators 10 are connected to the information processing server 100 in an accessable manner via the network by the terminals used for the business, for example. Each of the business operators 10 may include a body care device 30 or may provide manual body care without using the body care device 30.

The network 40 is a directed to a network for interconnecting the robot body care system with various devices including, for example, a wireless network or a wired network. Specifically, the network 40 may include a wireless LAN (WLAN) or a wide area network (WAN), integrated service digital networks (ISDNs), wireless LANs, long term evolution (LTE), LTE-Advanced, 4th Generation (4G), 5th generation (5G), code division multiple access (CDMA), WCDMA (registered trademark), and Ethernet (registered trademark), for example.

The network 40 is not limited to these examples, but may include, for example, the public switched telephone network (PSTN), Bluetooth (registered trademark), and Bluetooth Low Energy (registered trademark), optical communication lines, asymmetric digital subscriber line (ADSL), low-power wide-area network (LPWAN) lines, satellite communication networks, or any other network.

The network 40 may also include, for example, a narrow band IoT (NB-IoT) and enhanced machine type communication (eMTC). In addition, NB-IoT and eMTC are wireless communication methods for IoT, which are low-cost, low-power, and long distance communication network.

The network 40 may also be a combination of these. The network 40 may also include a plurality of different networks, which may be a combination of these examples. For example, the network 40 may include a wireless network with LTE and a wired network, such as an intranet, which is a closed network.

Here, as shown in FIG. 1, the robot body care system 1 includes a camera 50 that detects facial expressions and body movements of a user P during a treatment. The camera 50 is provided and used along with the body care device 30. The camera 50 captures images of the expressions of the user P and transmits it to the information processing server 100. This imaged data allows the information processing server 100 to determine whether the user P is in a comfortable state or an uncomfortable state. In addition, the camera 50 captures the body movements of the user P to acquire video data allowing the pulses and respiration rate of the user P to be determined from the video data.

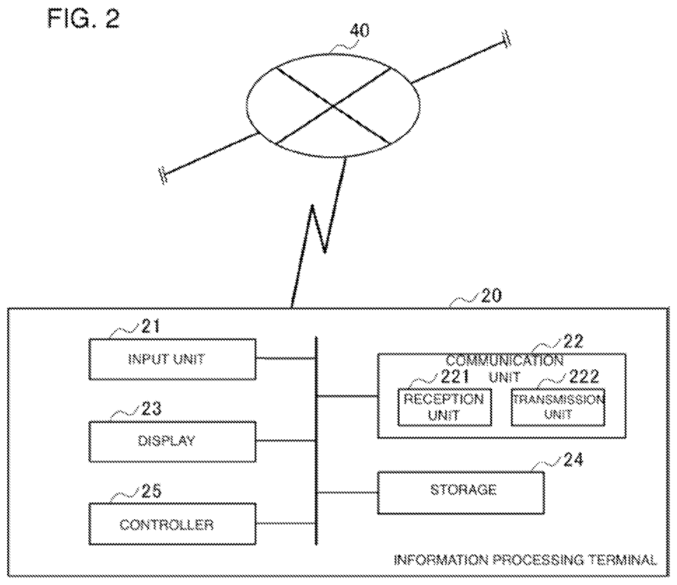

The information processing terminal 20 owned by the user P is directed to a terminal used by the user P that receives the body care, and is directed to, for example, a personal computer, a tablet, and a smart phone owned by the individual. In the illustrated example, although the information processing terminal 20 is directed to a smartphone, this is not limited thereto. The configuration of the information processing terminal 20 will be described with reference to FIG. 2. FIG. 2 is a block diagram illustrating the information processing terminal 20.

As shown in FIG. 2, the information processing terminal 20 includes an input unit 21, a communication unit 22, a display 23, a storage 24, and a controller 25. The input unit 21 serves to receive input from the user P. Although the input unit 21 may be embodied, for example, by hardware keys or a touch panel, it is not limited thereto. The input unit 21 communicates the input contents accepted from the user P to the controller 25.

The communication unit 22 is directed to a communication interface that serves to execute communication with the information processing server 100. The communication unit 22 includes a reception unit 221 and a transmission unit 222. The communication unit 22 executes communication with the information processing terminal 20 wirelessly, for example, in accordance with the Bluetooth Low Energy (registered trademark), Bluetooth (registered trademark), 3rd Generation (3G), 4th Generation (4G), and Long Term Evolution (LTE), but the communication standard is not limited thereto.

The display 23 is directed to a monitor that serves to display images and texts in accordance with the instructions from the controller 25. The display 23 may be embodied, for example, by an LCD, but is not limited thereto. The display 23 is capable of, for example, displaying historical information indicating the frequency with which the treatment command values are used, which will be described below.

The storage 24 is directed to a recording medium that serves to store various programs, data, and parameters needed for the operation of the information processing terminal 20. The storage 24 may be embodied by, for example, flash memory, SSD, and HDD, but it is not limited to thereto.

The controller 25 is directed to a processor that serves to control portions of the information processing terminal 20. The controller 25 controls the portions of the information processing terminal 20 by executing various programs stored in the storage 24. For example, the controller 25 transmits the evaluation information from the user P entered through the input unit 21 via the communication unit 22 to the information processing server 100.

The body care device 30 is driven and controlled by treatment command values described below, and is directed to a device that applies physical stimulation to the user P to perform the body care. As shown in FIG. 1, in the present embodiment, as the body care device 30, a configuration including robot hands 31 connected to corresponding distal ends of arms 32 will be described as an example. The mechanism of the body care device 30 need not be a hand structure as long as it includes a mechanism capable of applying physical stimulation to the user P to perform the body care.

The body care device includes at least a reception unit that receives treatment command values transmitted from the information processing server 100, a driving device (motor) that drives the robot hands 31 and the arms 32, and a driver device that generates a current value to be applied to the driving device on the basis of the treatment command values. In receiving the treatment command values, the treatment command values may be received via the network 40 or as media data via a storage medium such as a USB. In addition, a form may be employed in which the generated treatment command values may be input directly to the body care device.

A finger sensor is built into each finger tip of the robot hands 31 in the body care device 30. The finger sensor includes a piezoelectric sensor that measures pressure when the body of the user P is pressed, a temperature sensor that measures the temperature of the body of the user P, and a pulse sensor that measures the pulse of the user P. This allows the pressure at the time to be acquired while performing the body care on the user P, and also allows the temperature and pulse to be acquired as the state of the user P.

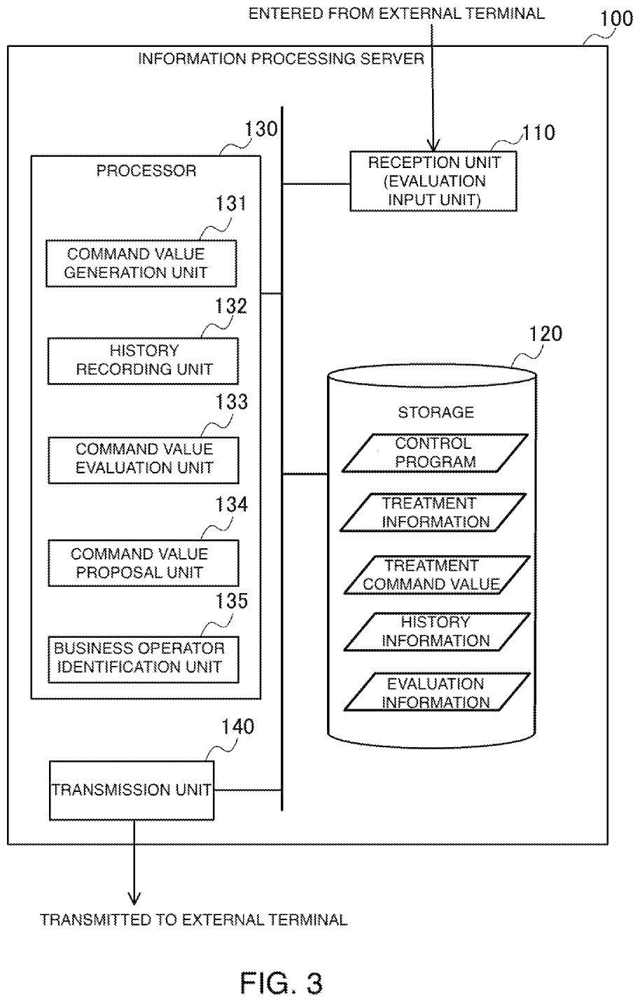

Next, the configuration of the information processing server 100 will be described with reference to FIG. 3. FIG. 3 is a block diagram illustrating the configuration of the information processing server 100 shown in FIG. 1. The information processing server 100 is directed to a processing device that allows the body care device 30 to provide body care that mimics manual body care. The information processing server 100, for example, uses the treatment method obtained from the business operator 10 as the treatment information.

Here, the treatment method is directed to a method of how to apply stimulation to the portions of the body of the user P. Specifically, the treatment method indicates a body portion of a user when the body care is performed, a stimulation method, a stimulation intensity, a stimulation time, a number of times and frequency of stimulation, an order when the body care is performed on plural portions, and a way of moving tools used for the body care.

As shown in FIG. 3, the information processing server 100 includes a reception unit 110, a storage 120, a processor 130, and a transmission unit 140. The reception unit 110 is directed to a communication interface that receives various data from an external terminal via the network 40 shown in FIG. 1.

The various data may include treatment information and evaluation information. The reception unit 110 receives, for example, treatment information indicating a treatment method for performing the body care on the user P from the business operator 10. The reception unit 110 may receive the treatment information not from the business operator 10 but from other organizations. For example, the reception unit 110 may machine learn a plurality of pieces of treatment information stored on the cloud 60 into a learning model, and then receive the treatment information derived by entering a predetermined condition from the external device.

Deep learning and boosting may be employed as the machine learning. For the machine learning, a learning model configured by a neural network may be used. Further, in place of performing the machine learning, the method may include a lookup table in which, for example, attributes such as the gender of the practitioner or physical characteristics of the practitioner are associated with the treatment information and stored, and the lookup table may be referred to so that the treatment information is selected. In addition, evaluation information from a plurality of users P configured by users P is entered into the reception unit 110. That is, the reception unit 110 serves as an evaluation input unit of the present disclosure.

The storage 120 serves to store various control programs and various data needed for the operation of the information processing server 100. The storage 150 may be embodied, for example, by various storage media such as HDD, SSD, and flash memory. The information processing server 100 embodies the functions to be embodied by executing the control programs stored in the storage 120. The functions here may include at least a reception function, a command value generation function, a body care function using the body care device 30, a treatment command value evaluation function, a command value proposal function, and a business operator identification function.

The storage 120 also stores treatment information as various data. The treatment information is directed to information acquired from the business operator 10, and is directed to information that indicates the specific contents of the body care by the treatment method. The treatment information is, for example, directed to information that indicates the contents of the body care performed by human hands.

The storage 120 also stores treatment command values as various data. The treatment command values refer to electrical signals as command values that are entered to the body care device 30 to cause the body care device 30 to imitate manual body care on the basis of the treatment information, and also refer to data generated by the command value generation unit 131 described below. The treatment command values are determined mainly by the treatment information and the mechanism of the body care device 30. A single treatment command value or multiple treatment command values may be used in a single body care session.

The storage 120 also stores history information as the various data. The history information is directed to information indicating a history of use of the treatment command values generated by the command value generation unit 131, and is directed to data that the history recording unit 132 described below stores in the storage 120. The history information is checked so that the number of times the plurality of treatment command values are used can be obtained, and the treatment command values that are popular to the plurality of users P (i.e., popular treatment methods) and the treatment command values that are not popular (i.e., not popular treatment methods) can be obtained.

The storage 120 also stores evaluation information as the various data. The evaluation information is directed to information in which each of the plurality of users P enters his or her own impressions as an evaluation of the treatment command values used after receiving the body care. The evaluation information may be, for example, a numerical rating on a scale of five, for example, or a direct input of impressions as comments.

The processor 130 is directed to a computer that controls the portions of the information processing server 100, such as a central processing unit (CPU), a microprocessor, an ASIC, and an FPGA, for example. The processor 130 includes a command value generation unit 131, a history recording unit 132, a command value evaluation unit 133, a command value proposal unit 134, and a business operator identification unit 135.

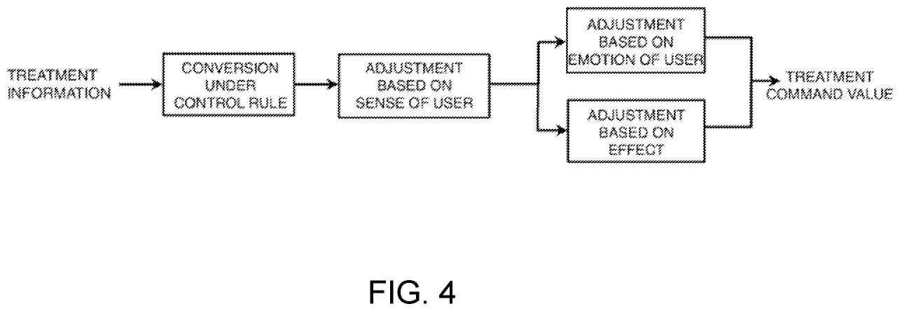

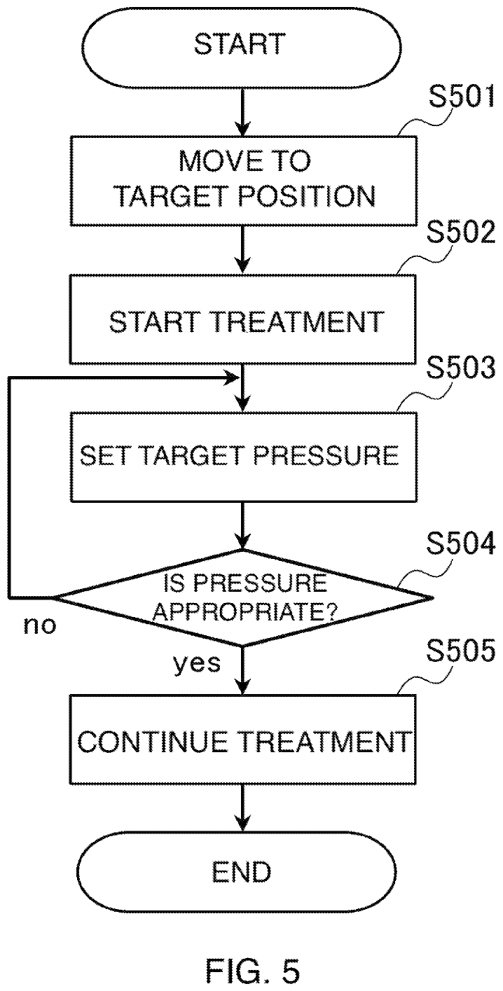

The command value generation unit 131 generates treatment command values to be entered to the body care device 3 from the treatment information entered into the reception unit 110. This point will be described in detail with reference to FIGS. 4 and 5. FIG. 4 is a schematic diagram illustrating an overview of the process by which the command value generation unit 131 generates treatment command values. FIG. 5 is a diagram illustrating a behavior of the body care by the body care device 30.

As shown in FIG. 4, the treatment information is converted into treatment command values through some steps. The first step is the conversion process under the control rule. This is set on the basis of the characteristics of the mechanism and control means of the body care device 30. That is, it is a problem how to drive the body care device 30 to embody the contents of the treatment to be performed manually with the body care device 30.

Here, the conversion method from the treatment information to the treatment command values will be described with specific examples. For example, a practitioner that performs body care manually while wearing measuring gloves, performs a treatment in accordance with the treatment method indicated in the treatment information. A plurality of accelerometers and finger pressure sensors are built into the measuring gloves. This allows the detection of the movement of the fingers when the practitioner performs the treatment, and the measurement of the coordinates, acceleration, and finger pressure when the robot hands 31 are driven. The coordinates, acceleration, and finger pressure measured as described above serve as the treatment information. The above described conversion method is only an example, and other methods may be employed to generate treatment command values from the treatment information.

Next, the treatment command values are adjusted by input on the basis of the senses of the user P. Here, the senses of the user P are directed to values recognized by her/him as values in which she/he feels comfortable when the finger pressure, finger position, and arm force, for example, of the robot hands 31 are changed, and may be adjusted in accordance with the preference of the user P.

This adjustment process is performed, for example, by the operator that operates the body care device 30 confirming the wishes of the user P, and from the reception unit 110, entering them to the information processing server 100.

In this case, the storage 120 may store the preferred command values of each user P therein. This adjustment process may be performed before or during the body care treatment.

Next, the treatment command values are adjusted by input on the basis of the emotions of the user P. Here, the emotions of the user P are directed to emotions of comfort or discomfort during the body care, and they are directed to emotions that unconsciously arise during the actual treatment. The emotions may be estimated by analysis by artificial intelligence from data such as body temperature and pulse obtained by the sensors described above and data such as respiration and facial expression obtained through the camera 50 to estimate optimal command values. When machine learning is performed by the artificial intelligence, for example, the optimal values may be calculated by a reinforcement learning model using an action value function. This adjustment may be performed during the treatment, or it may be reflected in the next treatment. Further, in the estimation of the emotions of the user P by the camera 50, cluster analysis may be performed by unsupervised learning, or the emotions may be estimated by classifying the image data, for example, by supervised learning. Moreover, the input on the basis of the emotions may also be performed by the operator on the basis of the responses from the users themselves.

Next, the treatment command values are adjusted by input on the basis of the effects. Here, the effects are directed to the change in the body temperature, and pulse, for example, when the treatment is actually performed. The presence or absence of the effects, i.e., whether the amount of change is large, may be confirmed by comparing the amount of change with the past amount of change at a time when the treatment was performed using the treatment command values. The optimal command values may then be estimated by the analysis by the artificial intelligence with reference to the finger pressure, finger position, and arm force, for example, employed in the highly effective treatment. In addition, when the machine learning is performed by the artificial intelligence, for example, the optimal values may be calculated by a reinforcement learning model using an action value function. This adjustment is performed during the treatment.

As shown in FIG. 5, when the body care device 30 is actually driven, first, the body care device 30 is moved to the target position (step S501). Next, the body care device 30 starts the treatment (step S502). A target pressure is then set as a target value of the finger pressure by the robot hands 31 (step S503). The processor 130 then determines whether the pressure is appropriate when the acupressure is performed at the set pressure. In this determination, adjustment is performed by inputs on the basis of the senses of the user P, emotions of the user P, and effects as described above.

If the pressure is not appropriate (no in step S504), the target pressure is set again (step S503). In contrast, if the pressure is appropriate (yes in step S504), the treatment is continued with the set pressure maintained (step S505). This process is performed not only for the acupressure force but also for all parameters needed for the robot hands 31 to perform the body care, such as the positions of the robot hands 31 and the force of the arms 32. As described above, the body care by the body care device 30 is performed in accordance with the command values generated by the command value generation unit 131.

As shown in FIG. 3, the history recording unit 132 records history information indicating the history in which the treatment command values generated by the command value generation unit 131 are used. The history recording unit 132 acquires log data indicating that the treatment command values have been used by the body care device 30 and causes the storage 120 to store the log data. This allows a history of what treatment command values, when, and with which body care device 30 have been used to be recorded.

The command value evaluation unit 133 evaluates the frequency of use of the treatment command values on the basis of the history information recorded in the history recording unit 132. The popularity of the treatment command values is evaluated on the basis of the contents of the evaluation information from the users P and the frequency of use. That is, the treatment command values that are used more frequently are determined to be more popular. In addition, the treatment command values that are highly evaluated in the evaluation information from the users P are also determined to be highly popular.

The command value proposal unit 134 proposes highly popular treatment command values to any one of the users P on the basis of the evaluation results by the command value evaluation unit 133. That is, by proposing the popular treatment command values, the popular treatment command values will be used more frequently.

The business operator identification unit 135 identifies, on the basis of the evaluation results by the command value evaluation unit 133, the business operator 110 that has entered the treatment information. That is, for example, the identification of the business operator 10 that has entered the popular treatment information allows the business operator 10 that has know-how regarding the highly popular treatment methods to be identified. Rewards in accordance with the number of times the treatment command values are used, for example, may be paid to the thus identified business operator. The transmission unit 140 is directed to a communication interface that transmits the treatment command values to the body care device 30 via the network 40 shown in FIG. 1.

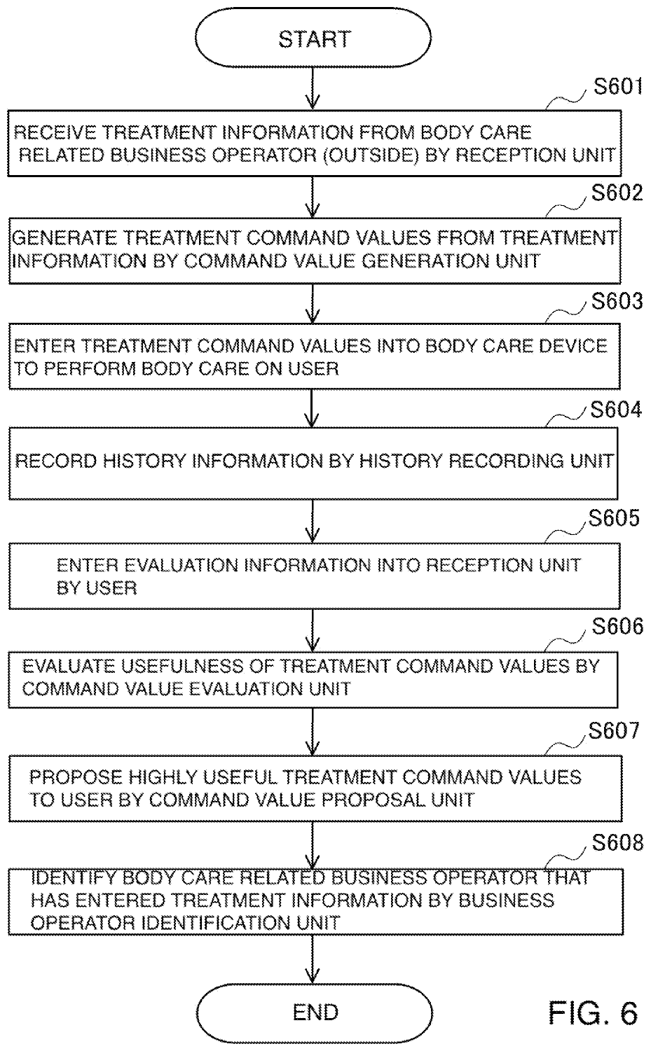

Next, with reference to FIG. 6, the procedure of processing in the robot body care system will be described. FIG. 6 is a diagram illustrating the processing flow of the robot body care system. As shown in FIG. 6, in the processing of the robot body care system, first, the reception unit 110 receives the treatment information from the business operator 10 (step S601: receiving step). In the receiving step, the business operator 10 enters the treatment information into the reception unit of the information processing server 100 from a terminal, for example, used for the business.

Next, the command value generation unit 131 generates treatment command values from the treatment information (step S602: command value generation step). The treatment command values are generated by the process described above. In this case, the various data acquired by the sensors, the previously generated treatment command values, and the comments thereon by the users P may be stored on the cloud 60 and used for generating the treatment command values.

Next, the treatment command values are entered into the body care device 30, and the body care is performed on the user P (step S603: the body care step). In the body care step, the body care device 30 performs a predetermined body care on the basis of the treatment command values. Next, the history recording unit 132 records the history information (step S604: history information recording step). In the history information recording step, the storage 120 stores the usage history of the respective treatment command values.

Next, the user P enters the evaluation information into the reception unit 110 (step S605: evaluation information input step). In the evaluation information input step, the user P enters his/her impressions and evaluations of the body care they received through the information processing terminal 20. Next, the command value evaluation unit 133 evaluates the popularity of the treatment command values (step S606: command value evaluation step). In the command value evaluation step, the popularity of the treatment command values is quantitatively evaluated, for example, by assigning a score.

Next, the command value proposal unit 134 proposes popular treatment command values to the user P (step S607: command value proposal step). In the command value proposal step, when highly popular treatment command values are proposed to the user P, the treatment command values to be proposed may be selected in accordance with the attributes such as the age and sex of the user P. The business operator identification unit 135 then identifies the business operator 10 that has entered the treatment information (step S608: business operator identification step). In the business operator identification step, the business operator 10 that has entered the highly popular treatment information may be awarded by paying a reward or displaying a ranking on the basis of the frequency of use.

As described above, according to the robot body care system according to the present embodiment, from the treatment information received by the reception unit 110 from the business operator 10, the command value generation unit 131 generates treatment command values. The body care device 30 then performs the body care on the user P on the basis of the treatment command values. This eliminates the need for the practitioner to directly perform the body care, and allows the practitioner's know-how regarding the body care to be quantitatively managed as treatment command values. This effectively utilizes the know-how possessed by various practitioners, simplifies the labor-intensive services such as body care services, and stabilizes the quality of the body care.

In addition, the history recording unit 132 records history information indicating a history in which the treatment command values have been used, and the command value evaluation unit 133 evaluates the frequency of use of the treatment command values on the basis of the history information. This determines the values that are used frequently as the treatment command values and identifies the highly popular treatment command values.

Also, the command value evaluation unit 133 evaluates the popularity of the treatment command values on the basis of the contents of the evaluation information from the users P and the frequency of use to make highly accurate determination on the popularity of the treatment command values on the basis of not only the frequency of use of the treatment command values but also the evaluation information entered from the users P.

In addition, the command value proposal unit 134 proposes highly popular treatment command values on the basis of the evaluation results by the command value evaluation unit 133 to any of the users P. Accordingly, the highly popular treatment command values are shared by a considerable number of the users P, improving the convenience of the users P.

This also allows the business operator identification unit 135 to identify the business operator 10 that has entered the treatment information on the basis of the evaluation results by the command value evaluation unit 133. Accordingly, for example, an operation is performed such that a reward is given to the business operator that has entered the highly popular treatment information identified by the business operator identification unit 135 to ensure the motivation of the business operator that has entered the treatment information.

The apparatus of the above embodiment is not limited to the above embodiment, and may be embodied by other methods. Various examples of variations will be described below.

For example, although the processor 130 in the information processing server 100 executes a control program, for example, to embody the above described embodiment, a logic circuit (hardware) or a dedicated circuit formed in an integrated circuit (IC) chip or a large scale integration (LSI), for example, in the apparatus may embody the above described embodiment in place. The functional units may be embodied by one or more integrated circuits, and the functions of the plurality of functional units may be embodied by a single integrated circuit. The LSI is sometimes referred to as a VLSI, Super LSI, and Ultra LSI, for example.

The above described control program may be recorded on a recording medium that is readable by the processor, and the recording medium may be a “non-temporary tangible medium,” such as a tape, disk, card, semiconductor memory, and programmable logic circuit. The above described control program may be supplied to the above processor via any transmission medium capable of transmitting the control program (such as a communication network or broadcast wave). The present disclosure may also be embodied in the form of a data signal embedded in a carrier wave, wherein the above described control program is embodied by electronic transmission.

The above control program may be implemented using, for example, a scripting language such as ActionScript or JavaScript (registered trademark), an object-oriented programming language such as Objective-C or Java (registered trademark), or a markup language such as HTML5.

Further, the configurations shown in the above embodiments and in the respective supplements may be combined as appropriate. Moreover, regarding the processing procedures, the order of the execution of the procedures may be changed or two processes may be executed in parallel as long as the results to be obtained are the same.

What is claimed is:

1. A robot body care system comprising:
a body care device that applies physical stimulation to a user to perform body care; and
an information processing server including:
a reception unit that receives from an outside a treatment method that indicates a way to stimulate portions of a body of a user when the body care is performed on the user as treatment information including information on coordinates, acceleration, and finger pressure when a robot hand is driven, the information being generated on the basis of results of measurement of a movement of fingers of a practitioner by a plurality of acceleration sensors and finger pressure sensors distributed on a measurement glove worn by the practitioner when the practitioner performs a treatment;
a command value generation unit that generates a treatment command value as an electrical signal on the basis of the treatment information entered via the reception unit; and
a transmission unit that transmits the treatment command value to the body care device, wherein
the body care device corresponds to the robot hand including a plurality of fingers to embody the body care, and
the treatment command value is adjusted by at least any of an input on the basis of a sense of the user in accordance with positions of the fingers of the robot hand, a finger pressure of the robot hand, and an arm force of the robot hand during the treatment of the body care performed on the user, and an input on the basis of an effect in accordance with a change in a body temperature and a pulse of the user caused by the treatment of the body care performed on the user.

2. The robot body care system according to claim 1, wherein
each finger tip of the robot hand includes, as finger sensors, a piezoelectric sensor that measures a pressure when the body of the user is pressed, a body temperature sensor that measures the body temperature of the user, and a pulse sensor that measures the pulse of the user, and
the command value generation unit generates the treatment command value on the basis of:
input on the basis of a sense of the user in accordance with a position of the fingers of the robot hand, a finger pressure of the robot hand, and an arm force of the robot hand during a treatment of the body care performed on the user;
a first optimum value calculated on the basis of a learning model and an emotion of the user estimated on the basis of the effect in accordance with the body temperature and pulse of the user during the treatment of the body care performed on the user; and
a second optimum value calculated on the basis of a learning model and a past treatment command value at which a change in the body temperature and pulse of the user is significant before and after the treatment of the body care performed on the user, and
the treatment command value is adjusted during the treatment of the user.

3. The robot body care system according to claim 1, wherein the treatment command value is adjusted for use in next and subsequent treatments.

4. The robot body care system according to claim 1, further comprising:
a history recording unit that records history information indicating a history in which the treatment command values generated by the command value generation unit are used;
an evaluation input unit in which evaluation information indicating an evaluation of the treatment command value utilized by the user is entered; and
a command value evaluation unit that evaluates popularity of the treatment command value on the basis of the history information recorded in the history recording unit and the evaluation information entered in the evaluation input unit.

5. The robot body care system according to claim 4, wherein the users enter the evaluation information in the evaluation input unit.

6. The robot body care system according to claim 4, further comprising a command value proposal unit that proposes a popular treatment command value to one of the users on the basis of an evaluation result by the command value evaluation unit.

7. The robot body care system according to claim 6, wherein
the reception unit receives the treatment information from a body care related business operator, and
the robot body care system further includes a business operator identification unit that identifies a body care related business operator that has entered a highly popular piece of the treatment information as an excellent business operator on the basis of the evaluation result by the command value evaluation unit.

8. The robot body care system according to claim 7, wherein a reward is paid to the excellent business operator identified by the business operator identification unit.

9. A robot body care method causing a computer to execute the steps of:
receiving from an outside a treatment method that indicates a way of applying stimulation to portions of a body of a user when a body care is performed on the user as treatment information including information on coordinates, acceleration, and finger pressure when a robot hand is driven, the information being generated on the basis of results of measurement of a movement of fingers of a practitioner by a plurality of acceleration sensors and finger pressure sensors distributed on a measurement glove worn by the practitioner when the practitioner performs a treatment;
generating a treatment command value as an electrical signal on the basis of the treatment information entered in the receiving step;
transmitting the treatment command value to a body care device that applies physical stimulation to the user to perform the body care; and
performing the body care by the body care device using the treatment command value, wherein
the body care device corresponds to the robot hand including a plurality of fingers that mimic manual body care to embody the body care, and
the treatment command value is adjusted by at least one of an input on the basis of a sense of the user such as a preference of the user in accordance with a position of the fingers of the robot hand, a finger pressure of the robot hand, and an arm force of the robot hand during a treatment of the body care performed on the user; and an input on the basis of an effect in accordance with a change in a body temperature and pulse of the user.

10. A non-transitory computer readable medium storing therein a robot body care program causing a computer to execute the functions of:
receiving from an outside a treatment method that indicates a way of applying stimulation to portions of a body of a user when a body care is performed on the user as treatment information including information on coordinates, acceleration, and finger pressure when a robot hand is driven, the information being generated on the basis of results of measurement of a movement of fingers of a practitioner by a plurality of acceleration sensors and finger pressure sensors distributed on a measurement glove worn by the practitioner when the practitioner performs a treatment;
generating a treatment command value as an electrical signal on the basis of the treatment information entered in the receiving function;
transmitting the treatment command value to a body care device that applies physical stimulation to the user to perform the body care; and
performing the body care by the body care device using the treatment command value, wherein
the body care device corresponds to the robot hand including a plurality of fingers that mimic manual body care to embody the body care, and
the treatment command value is adjusted by at least any of an input on the basis of a sense of the user in accordance with positions of the fingers of the robot hand, a finger pressure of the robot hand, and an arm force of the robot hand during the treatment of the body care performed on the user, and an input on the basis of an effect in accordance with a change in a body temperature and a pulse of the user caused by the treatment of the body care performed on the user.
